(19) United States
(12) Patent Application Publication
(10) Pub. No.: US 2026/0034032 A1
(43) Pub. Date: Feb. 5, 2026

(54) SOLID DISCRETE PARTICLES AND PERSONAL CARE COMPOSITION CONTAINING THE SAME

(71) Applicant: THE PROCTER & GAMBLE COMPANY, Cincinnati, OH (US)

(72) Inventors: Martin Chang Shou LEE, Singapore (SG); Gemma Zoe BRAGANZA, Singapore (SG); Shikhar GUPTA, Queenstown (SG)

(21) Appl. No.: 19/284,934

(22) Filed: Jul. 30, 2025

Related U.S. Application Data
(60) Provisional application No. 63/679,368, filed on Aug. 5, 2024.

(51) Int. Cl. A61K 8/02; A61Q 5/00; A61Q 5/12; A61Q 19/10

(52) U.S. Cl. CPC A61K 8/0283; A61Q 5/008; A61Q 5/12; A61Q 19/10; A61K 2800/31; A61K 2800/87

(57) ABSTRACT

A solid particle for use in a personal care composition. The particle includes a C₁₂-C₂₂ fatty alcohol; an amidoamine cationic surfactant, an oil soluble acid having an AlogP of −1.2 or greater, less than 50% water and, optionally, a benefit agent. A plurality of the solid, discrete particles can be added to an aqueous carrier to form a personal care composition, or to a pre-existing personal care composition to provide a benefit. The particles have a relatively high melt point to reduce agglomeration and sintering during shipping and storage.

### CROSS REFERENCE TO RELATED APPLICATIONS

This application claims the benefit, under 35 U.S.C. § 119(e), to U.S. Provisional Application No. 63/679,368, filed Aug. 5, 2024, the entire disclosure of which is fully incorporated by reference herein.

### FIELD

The present disclosure relates generally to solid gel network particles that deliver a benefit to a composition in which the particles are dispersed. More specifically, the present disclosure relates to discrete solid gel network particles comprising a cationic surfactant, a fatty alcohol and an oil soluble acid with an AlogP of −1.2 or greater.

### BACKGROUND

A variety of approaches have been developed to deliver cleaning and conditioning benefits to skin and hair. For example, a common method of conditioning hair involves applying a hair conditioning composition that delivers a conditioning agent to hair or to a target portion of hair. Some examples of known conditioning agents include cationic surfactants, cationic polymers, fatty alcohols, botanical oils, and silicone compounds. Cationic conditioning agents typically include a cationic head portion, which electrostatically bonds to exposed cysteine residues in the hair cuticle, and a long hydrophobic tail portion that lies flat along the surface of the hair to provide a lubrication, softness, moisturization, anti-static, anti-frizz or other conditioning benefit. While conventional conditioning agents generally provide suitable conditioning benefits for at least some users, there is a need for flexibility in the level and/or type of conditioning benefit provided. The same can also be said for the benefit(s) provided by other personal care compositions such as shampoos and body washes.

U.S. Pat. No. 11,058,620 discloses an aqueous personal care composition that contain discrete particles for providing more flexibility in the product composition rheology, improved stability in the product composition, improved deposition of benefit agents, and/or improved visual/aesthetic appearance. The particles exemplified in U.S. Pat. No. 11,058,620 are a mixture of a cationic surfactant (stearamidopropyl dimethylamine (SAPDMA) and/or behenyl trimethyl ammonium sulfate (BTMS)), a high-melting point fatty alcohol (stearyl and cetyl alcohol) and a benefit agent (colorants or conditioning agents). These particles can be thought of as conditioning beads.

Conditioning beads, such as those disclosed in U.S. Pat. No. 11,058,620, are typically manufactured by a supplier and shipped to a manufacturer for incorporation into a personal care composition. The particles may be stored in a warehouse or the like before and/or after shipping. During shipping and/or storage, the particles may experience a wide range of environmental conditions (e.g., temperature and humidity fluctuations). When conventional conditioning beads are exposed to high temperatures (e.g., >40° C.), they may soften or melt, resulting in compressed or agglomerated particles that are more difficult to incorporate into a personal care composition than discrete, free flowing particles. In particular, free flowing particles are more easily dispersed in a personal care composition and more evenly distributed throughout the product.

Accordingly, it would be desirable to provide discrete particles that exhibit reduced sintering and agglomeration during transport and storage.

### SUMMARY

The present disclosure is directed to a solid, discrete particle, comprising: a C₁₂-C₂₂ fatty alcohol; an amidoamine cationic surfactant; an oil soluble acid having an AlogP of −1.2 or greater; an optional benefit agent; and less than 50% water.

### BRIEF DESCRIPTION OF THE DRAWINGS

FIG. 1 is an illustration of a solid particle and a swollen particle.

FIG. 2 shows the relationship between the AlogP of the acid and the DSC Onset Melt Temperature of a discrete particle and the relationship between the AlogP of the acid and the Creep-recovery value of the particle.

### DETAILED DESCRIPTION

Conditioning beads in the form of solid discrete particles are known. However, these conventional particles can soften or melt during transport and/or storage as a result of high temperatures or other environmental conditions, which makes the particles are harder to process, especially if they experience agglomeration or sintering (sometimes referred to as “caking”). It has now been discovered that solid gel network particles formulated with a specific combination of fatty alcohol, amidoamine cationic surfactant and oil soluble acid exhibit reduced sintering when the particles are under pressure and compaction. In one example, a solid gel network particle made with a stearamidopropyl dimethylamine (SAPDMA) cationic surfactant, C₁₂₋₂₂ fatty alcohol (e.g., stearyl, cetyl and/or behenyl), and an oil soluble acid having an octanol/water partition coefficient (AlogP) of greater than −1.2 exhibits unexpectedly reduced sintering and agglomeration. This is unexpected because it was previously believed that all combinations of SAPDMA, C₁₂₋₂₂ fatty alcohols and an acid counterion would generally yield similar results.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“AlogP” refers to a well-known method for estimating the octanol/water partition coefficient (log P) of a material. A description and explanation of the method can be found, for example, in Ghose, et al. (1998); “Prediction of Hydrophobic (Lipophilic) Properties of Small Organic Molecules Using Fragmental Methods: An Analysis of AlogP and ClogP Methods”. Journal of Physical Chemistry A. 102 (21): 3762-3772; and Ghose, et al., “Atomic Physicochemical Parameters for Three-Dimensional Structure-Activity Relationships I. Partition Coefficients as a Measure of Hydrophobicity” J. Comput. Chem. 1986, 7, 565-577.

“Base composition” refers to a composition to which the discrete particles are added. A base composition can be a personal care composition such as a shampoo, conditioner or body wash, or a base composition can be a carrier such as water, with or without other ingredients.

“Charge density” (“CD”) means the ratio of positive charges on a polymer to the molecular weight of the polymer.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Gel network” refers to a lamellar or vesicular solid crystalline phase in an aqueous personal care composition. The gel network comprises at least one fatty amphiphile, at least one surfactant and a suitable solvent (e.g., water), where the fatty alcohol and surfactant within this phase are arranged in multi-lamellar vesicles and/or lamellar sheets. “Solid crystalline” refers to the structure of the lamellar or vesicular phase that forms at a temperature below the melt transition temperature (i.e., the chain melt temperature) of the layer in the gel network (typically 27° C. or higher). The melt transition temperature may be measured by differential scanning calorimetry.

“Molecular weight” refers to the weight average molecular weight expressed as grams/mole, unless otherwise stated. Molecular weight is measured using industry standard method, gel permeation chromatography (“GPC”).

“Solid,” when referring to a particle, means that the particle does not conform to the shape of the container in which it is held at 25° C.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Swollen,” and variations thereof, refer to a discrete particle that has absorbed a sufficient quantity of water to exhibit a Compression Force of 50 grams-force (g-f) or less, according to the Compression method described in more detail below.

“Water soluble” means that the material is soluble in water in the composition. In general, the material should be soluble at 25° C. at a concentration of 0.1% by weight of the water solvent, alternatively at 1%, alternatively at 5%, and alternatively at 15%.

### Discrete Solid Particles

The discrete solid particles herein include a C₁₂-C₂₂ fatty alcohol, an amidoamine cationic surfactant, an oil soluble acid with a partition coefficient (AlogP) of −1.2 to 1.1 and, optionally, a benefit agent. In some instances, the particles may be prepared by co-melting a high melting point fatty alcohol (e.g., cetyl alcohol, stearyl alcohol and/or behenyl alcohol), stearamidopropyl dimethylamine (SAPDMA) and an oil-soluble acid, along with any optional ingredients desired (e.g., colorants, perfumes or hair benefit agents), and then cooling the mixture to solidify it. The cooled mixture may be formed into particles, for example, by spray drying (aerosolization), milling, agglomeration, sieving, rotoform, extrusion, grinding, stamping, cutting, spheronization, hot pour molding, combinations of these and the like. Some non-limiting examples of making fiber-shaped particles are disclosed in U.S. Pat. No. 11,351,094.

The fatty alcohol, cationic surfactant and oil-soluble acid may, collectively, be present at 10% to 100% based on the weight of the particle (e.g., 20% to 100%, 40% to 100%, 60% to 100% or 50% to 100%). The total liquid level in the particle or the pre-melt mixture of ingredients is less than 50%, 40%, or 30% by weight of the mixture composition, The cationic surfactant and the fatty alcohol may be present at a weight ratio of cationic surfactant to fatty alcohol of 20:1 to 1:20 (e.g., 10:1 to 1:10; 8:1 to 1:8; 6:1 to 1:6, 5:1 to 1:5, 4:1 to 1:4, or 3:1 to 1:3).

The discrete particle contains less than 50% water (e.g., less than 30%, 25%, 15%, 10%, 8%, 5%, or even less than 3% water) by weight of the particle. In some instances, the particle is anhydrous (i.e., 0% water). Prior to incorporation into a base composition, the discrete particle is dry (i.e., are not suspended in a liquid carrier) and solid.

The discrete particle can have a particle size of 1 μm to 100 mm. The particles size may vary widely depending the desired use of the particle. For example, when the particle is intended to be added to a base composition (e.g., shampoo, conditioner or body wash), the particle size may be 1 μm to 20 mm (e.g., 10 μm to 5 mm, 50 μm to 2 mm, 200 μm to 1 mm, or 500 μm to 1 mm). In some instances, a particle size of 500 μm to 2 mm may be particularly suitable. In another example, larger particles (e.g., 3 mm to 100 mm, 10 mm to 90 mm, 15 mm to 80 mm or 15 mm to 70 mm) may be mixed with a relatively large volume (e.g., 10 L, 25 L, 50 L, 100 L, 500 L or more) of a base composition or water as part of a scaled making process. In this example, at least some of the particles may transform into a continuous gel network when suitably mixed with the base composition or water. The particle size of the solid discrete particles can be measured using conventional methods.

The particles herein can be in any shape, for example, spherical, hemispherical, rectangular, diamond, fiber-like, flake-like, or irregular. Fiber-shaped particles can have an aspect ratio of 3 or more (e.g., greater than 4, 5, 6, 7, 8, 9 or even greater than 10). Flake-like particles generally appear flat or 2-dimensional and have an aspect ratio of less than 3. As used herein, aspect ratio refers to the ratio of longest dimension (length) to shortest dimension (width) of a particle. Various methodologies known in the art can be used to control the particle size and/or shape of the particles.

The discrete particles have an Onset Melt Temperature of 40° C. or greater (e.g., greater than 41° C., 42° C., 43° C., 44° C., or 45° C.), which can be determined according to the method described in more detail below. It is important to have an Onset Melt Temperature of 40° C. or more so that the particles do not softening or melt during shipping or storage.

The dry, solid particles herein exhibit a Compression Force of greater than 50 grams-force (g-f) according to the Compression method described in more detail below. The solid particles herein have a Creep-Recovery value of −0.003 or greater (e.g., −0.0028, −0.0026, −0.0024, −0.0022, or even −0.0020 or greater). Creep-recovery value can be determined according to the method described in more detail below. Particles that resist compression and deformation under force are desired.

The discrete particles exhibit good resistance to caking. Caking occurs when the particles undergo agglomeration or sintering and form larger solid masses, often as a result of high temperature, pressure and/or humidity. Agglomerated or sintered particles are harder to process into personal care products and may result in undesirable features such as grittiness, visible clumps of particles in the composition, and poor conditioning.

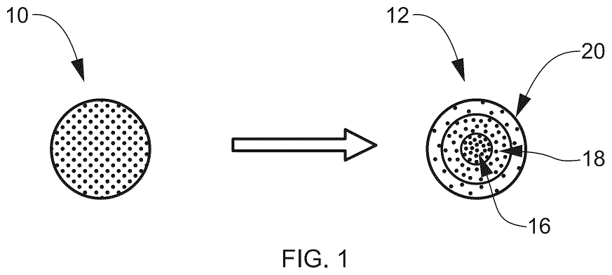

When placed in water or an aqueous composition, a dry discrete particle herein absorbs water and swells. FIG. 1 shows an illustration of a dry particle 10 and a swollen particle 12. The dry particle 10 contains a relatively low amount of water (e.g., less than 10%) distributed throughout the particle 10. However, the swollen particle 12 illustrated in FIG. 1 has a semi-hydrated gel phase 18, a hydrated gel phase 20 and an unhydrated solid phase 16, depending on how much water the particle absorbs. The swollen particles can have a particle size that is 50% greater than their dry counterpart (e.g., greater than 75%, 100% 150%, 200% or more). Swollen particles and/or the hydrated and semi-hydrated phases thereof may exhibit a compression force of 50 g-f or less (e.g., less than 40 g-f, 30 g-f, 20 g-f, 10 g-f, 5 g-f or even less than 0.5 g-f), according to the Compression method.

The particles herein are generally not coated or encapsulated (e.g., with a polymer), but embodiments in which the particles are coated or encapsulated are contemplated herein. The particles are not in the form of an emulsion (e.g., water-in-oil or oil-in-water) before or after swelling, which is evidenced by the lack of birefringence exhibited by the particles. The particles may be free of or substantially free of swellable silicone elastomers and swellable thickening polymers.

The particles herein may be made using conventional methods known in the art. Some non-limiting examples of methods to make discrete particles are disclosed in U.S. Pat. Nos. 11,931,441 and 11,058,621.

The particle herein may be used in a personal care product such as a shampoo, conditioner, or body wash that comprises an aqueous carrier. For example, a multitude of particles may be added to a personal care composition to modify a property or benefit of the composition (e.g., enhance or customize the amount of conditioning agent in the composition). Additionally or alternatively, a multitude of discrete particles may be added to an aqueous carrier (e.g., water), along with other optional ingredients, to form a personal care product such as a hair conditioner.

### High Melting Point Fatty Compound

The discrete particles herein include a high melting point fatty compound. The high melting point fatty compound may be selected to provide conditioning benefits such as friction reduction during conditioner application, case of spreading, case of detangling and good product stability. The high melting point fatty compound useful herein can have a melting point of 25° C. or higher, 9 (e.g., more than 40° C., 45° C., or even more than 50° C.), but typically less than 90° C. (e.g., less than 80° C., 70° C. or even less than 65° C.). It is believed, without being limited by theory, that a melting point below 25° C., or even below 40° C. in some instances, can introduce instability into the composition and/or the gel matrix (e.g., visible phase separation). On the other hand, fatty compounds with melt temperatures that are too high can be difficult to process during manufacturing. The high melting point fatty compound can be a single compound or a blend or mixture of two or more high melting point fatty compounds. When a blend or mixture is selected, the melting point means the melting point of the blend or mixture. Thus, a fatty compound with a melting point of less than 40° C. can be used form a discrete particle herein when it is combined with a fatty compound that yields a combined melt temperature of 40° C. or more.

Some non-limiting examples of high melting point fatty compounds that may be suitable for use herein are fatty alcohols, fatty acids, fatty alcohol derivatives, fatty acid derivatives and combinations thereof. Additional examples can be found in the International Cosmetic Ingredient Dictionary, Fifth Edition, 1993, and CTFA Cosmetic Ingredient Handbook, Second Edition, 1992.

Among the variety of high melting point fatty compounds, fatty alcohols are particularly suitable for use in present particles, especially those having 14 to 30 carbon atoms (e.g., 16 to 24 carbon atoms, 16 to 22 carbon atoms and 18 to 22 carbon atoms). The fatty alcohols may be saturated or unsaturated and straight or branched chain alcohols. Particularly suitable examples of fatty alcohols are cetyl alcohol (having a melting point of about 56° C.), stearyl alcohol (having a melting point of about 58-59° C.), behenyl alcohol (having a melting point of about 71° C.), and mixtures thereof. In one example, the fatty alcohol is a mixture of cetyl alcohol and stearyl alcohol. In this example, the weight ratio of cetyl alcohol to stearyl alcohol is 1:9 to 9:1 (e.g., 1:4 to 4:1, or 1:2.3 to 2.3:1).

### Cationic Surfactant

The discrete particle herein includes a cationic surfactant at an amount of 1% to 95% (e.g., 2-90%, 3-85%, 4-75%, 5-60%, 10-50%, 15-40% or 20-35%) based on the weight of the particle. The cationic surfactant can be a mono-long alkyl amine, a mono-long alkyl amidoamine or a combination thereof. Suitable mono-long alkyl amines and mono-long alkyl amidoamines include a long alkyl chain of 12 to 30 carbon atoms (e.g., 16 to 24 carbon atoms or 18 to 22 carbon atoms). Primary, secondary, and tertiary fatty amines may be used.

Particularly useful are tertiary amidoamines having an alkyl group of 12 to 22 carbons such as, for example, stearamidopropyldimethylamine (SAPDMA), stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine (BAPDMA), behenamidopropyldiethylamine, behenamidoethyldiethylamine, behenamidoethyldimethylamine, arachidamidopropyldimethylamine, arachidamidopropyldicthylamine, arachidamidoethyldiethylamine, arachidamidoethyldimethylamine, diethylaminoethylstearamide. Additional examples are disclosed in U.S. Pat. No. 4,275,055. A particularly suitable example of a tertiary amidoamine suitable for use in the present particles is SAPDMA.

### Oil Soluble Acid

Amidoamine cationic surfactants such as SAPDMA are uncharged and require an acid to protonate the surfactant, thereby making it more soluble in water. Indeed, the addition of the acid transforms the amidoamine and fatty alcohol mixture into a creamy composition in water, which is suitable for use as a personal care composition or composition base. However, f a suitable acid is not provided, then the amidoamine and fatty alcohol will remain a solid wax mixture in water, which is generally unsuitable for use in a personal care composition.

The discrete particle herein includes an oil soluble acid with an AlogP, of greater than −1.2, but typically less than 6.5 (e.g., −1.2 to 1.2, −1.1 to 1.1, −1.05 to 1.0, −1.05 to 0.9, −1.05 to 0.8, −1.05 to 0.7, −1.05 to 0.6, −1.05 to 0.5, and −1.05 to 0.4). If the acid is not soluble in oil, it does not improve the creep-recovery property of the discrete solid particle, and the acid may remain as a separate phase of crystalline form in the solid particle, thereby restricting the ability of the acid to transform the amidoamine and fatty alcohol mixture into a creamy composition in water. If the AlogP is not within the desired range, then the particles may have an Onset Melt Temperature that is too low and/or the particles may undergo sintering (e.g., if the acid causes too much moisture absorption). It has been found that even slight departures of the AlogP outside the desired range can undesirably impact the Onset Melt Temperature of the particle.

It has been surprisingly discovered that selecting an oil soluble acid with an AlogP of −1.2 to 1.1 can result in particles with a melt temperature of greater than 40° C. and a Creep-Recovery value of more than 0.0024. Table 1 below lists some examples of organic acids that can be used in personal care products along with their respective AlogP values and oil solubility. However, not all of the acids listed in Table 1 are suitable for use in the present particles. For example, lactic acid may be oil soluble, but it is relatively hygroscopic and typically supplied as a liquid instead of a powder, which is undesirable.

TABLE 1

| | AlogP | Oil Soluble |
|---|---|---|
| Citric acid | −1.32 | No |
| Glutamic acid | −3.46 | No |
| Lactic acid | −0.39 | Yes |
| Malic acid | −1.01 | Yes |
| Mandelic Acid | 0.84 | Yes |
| Maleic acid | 0.04 | Yes |
| Salicylic Acid | 1.22 | Yes |
| Succinic Acid | −0.36 | Yes |
| Cinnamic Acid | 1.93 | Yes |
| Azelaic acid | 1.92 | Yes |
| Glyoxalic acid | −0.41 | Yes |

### Additional Ingredients

The discrete particle, optionally, includes one or more additional ingredients to provide a formulation or performance benefit to the particle and/or a product containing the particle. For example, the additional ingredient may help provide: more flexibility in the product composition rheology, improved stability in the product composition, improved deposition of a benefit agent on hair or skin (e.g., hair or skin conditioning agent, moisturizing agent, anti-dandruff agent, vitamin, mineral, or enzyme), improved visual/aesthetic appearance of the particle and/or product containing the particle and/or a fragrance. An additional ingredient may be present in the particle at 0.01% to 50% (e.g., 0.05% to 40%, 0.1% to 35%, 0.5% to 30%, 1% to 25%, 3% to 20%, 5% to 15% or about 10%), based on the weight of the particle.

It may be desirable to formulate the particles to provide a predetermined amount of a particular ingredient to the aqueous base composition when the particles are added thereto (e.g., 0.05% to 30%, 0.1% to 15%, 0.5% to 10%, or 1% to 7%), based on the weight of the composition. It is important to tailor the amount of benefit agent present in the base composition so as not to deteriorate the benefit of the aqueous base composition, especially any cleansing or hair condition agents (e.g., surfactants or high melting point fatty compounds).

In some instances, the particles may include a secondary surfactant. The secondary surfactant is separate from and in addition to the amidoamine cationic surfactant component, and it may be the same or a different type of surfactant selected for use as a detersive surfactant in a product containing the particles. The secondary surfactant can be selected from anionic, cationic, amphoteric, zwitterionic and nonionic surfactants. Some non-limiting examples of surfactants are disclosed in US 2006/0024256. In some instances, it may be desirable to select a secondary surfactant that contributes to obtaining a higher melt transition temperature (e.g., greater than 40° C.). For example, the secondary surfactant may have a hydrophobic tail with a chain length of 16 to 22 carbon atoms selected from alkyl, alkenyl (containing up to 3 double bonds), alkyl aromatic, and branched alkyl groups.

The additional ingredient may be a conditioning agent (e.g., silicone polymer, cationic polymer or non-ionic polymer), a coloring agent such as FD&C or D&C dyes, pigments, mica or titanium dioxide, or any other benefit agent commonly found in hair and skin care products. Some nonlimiting examples of polymeric conditioning agents are described in US. A wide variety of other additional components can be formulated into the present compositions. These include: vitamins or pro-vitamins such as panthenol, panthenyl ethyl ether, hydrolysed keratin, proteins, plant extracts, and nutrients; preservatives such as benzyl alcohol, methyl paraben, propyl paraben and imidazolidinyl urea; pH adjusting agents, such as sodium hydroxide, sodium carbonate; perfume; ultraviolet and infrared screening and absorbing agents such as benzophenones; sequestering agents, such as disodium ethylenediamine tetra-acetate; antidandruff agents such as zinc pyrithione and piroctone olamine; organic and inorganic salts such as sodium chloride.

The additional ingredient may be an ingredient that is incompatible with at least one other ingredient in the particle (incompatible agent) such as, for example, solid minerals or chemical substances that have high ionic strength and/or high surface charge and tend to cause agglomeration and/or crystallization. Some non-limiting examples of incompatible agents include acids, amino acids, mica, salicylic acid, and metal pyrithione (e.g., zinc pyrithione with or without ionic polymer coating or dispersion), and organic oil materials that are highly interactive with a gel network component (e.g., hexyldecanol and isostearyl isostearate). Other examples of incompatible ingredients include surfactants that interact with one another such as anionic and cationic surfactants.

### Personal Care Product

The discrete particles herein may be added to a base composition (e.g., water) to form a personal care product, or the particles may be added to a personal care composition (e.g., shampoo, conditioner or body wash) to modify a property or benefit of the personal care composition. In some aspect, the composition to which the particles are added (“receiving composition”) may include a pre-formed dispersed gel network phase, for example, as described in US 2018/0311135. The pre-formed gel network may be present in an amount of 0.05% to 14% (e.g., 0.5% to 10% or 1% to 8%).

When added to a receiving composition, the solid discrete particles swell by absorbing water from the aqueous carrier and/or gel network. When the discrete particles contain water before being added to the receiving composition, the water content of the particles should be limited to less than 50% (e.g., less than 25%, 15%, 10%, or 8%), based on the weight of the particle. The particles are typically saturated and attain their maximum swollen particle size within 3 days or, in some instances, up to a week. The physical properties and benefits of the particles may change during swelling, but the swollen particles are generally stable after 3 weeks and remain stable in the composition for a month or longer (e.g., more than 3 months, 6 months, 12 months or even 24 month).

In addition to an aqueous carrier, the receiving composition may include other ingredients commonly used in composition of the type such as, for example, detersive surfactants, co-surfactants, water-miscible solvents, conditioning agents, hair or skin benefit agents, emulsifiers, thickeners, preservatives, chelants, structurants, suspending agents, colorants, and fragrances. Some non-limiting examples of additional ingredients found in personal care compositions can be found in U.S. Pat. No. 11,786,447 and US 2018/0098923. Surfactants, when present, may be anionic, amphoteric, zwitterionic, cationic or non-ionic, for example, as disclosed in US 2018/0098923. Conditioning agents that enhance deposition efficiency or glossiness of hair, when present, can include: polymeric silicone conditioning agents and organic conditioning oils, for example, as disclosed in U.S. Pat. No. 11,786,447; crosslinked cationic polymers, for example, as disclosed in U.S. Pat. No. 5,674,478; and any other conditioning agents known in the art, for example, as disclosed in U.S. Pat. Nos. 4,507,280, 4,663,158, 4,197,865, 4,217,914, 4,381,919, and 4,422, 853.

The receiving composition may be a shampoo that includes a fatty amphiphile (e.g., fatty alcohol, fatty ester, fatty acid, fatty amide) and a detersive surfactant (i.e., an anionic surfactant that provides a cleaning benefit). In such a composition, the discrete particle, when hydrated, may be tailored to form a gel network in the shampoo composition. The fatty amphiphile may be present at 0.05% to 20% (e.g., 0.5% to 10% or 1% to 8%), by weight of the shampoo composition. The weight ratio of fatty amphiphile to co-surfactant in the gel network component is greater than 1:9 (e.g., greater than 1:5, 1:2, 1:1) but typically less than 100:1 (e.g., less than 50:1 or 10:1).

### METHODS

### Creep-Recovery

This method can be used to determine the rate of deformation or compression under pressure of a single solid particle using a controlled-axial stress rheometer capable of sample temperature control, such as a Discovery™ HR-2 brand rheometer from TA Instruments, or equivalent, which uses a Peltier cooler and resistance heater combination.

The rheometer is operated in a parallel plate configuration with 8-mm stainless steel parallel-plate tooling. The rheometer testing temperature is set to 25° C. A solid particle is trimmed to a circular shape of approximate 4.3 mm diameter and gently loaded onto a Peltier plate with 8 mm plate geometry. Pre-clamping procedure is initiated by setting Compression Axial force to 0.2 Newton with gap setting within 200 μm of sample height. Sample is then equilibrated at 40° C. for 600 seconds, followed by further equilibrating at 40° C. for 1800 seconds with 0.2 Newton constant axial force. The test commences with the rheometer increasing the axial force to 0.64 Newton and maintaining the axial force for 1800 seconds.

For each Creep-recovery test, the strain amplitude measured during constant axial force of 0.64 Newton is analyzed by plotting strain amplitude (percent strain, vertical axis) versus Time (seconds, horizontal axis). Solid deformation or compression is defined by measuring the change of strain amplitude in the last 900 seconds when constant axial force is 0.64 Newton. The change in strain amplitude is calculated by measuring the slope of a straight line of strain amplitude datapoints, extracted using TRIOS software (provided by TA instrument) and is applicable for other equivalent rheology software.

### Onset Melt Temperature

The melt transition behavior and temperature for the gel network may be determined using a Discovery™ 2500 brand differential scanning calorimeter (DSC) from TA Instruments or equivalent, according to the following method. Approximately 5 mg of the solid particle is placed into a Tzero aluminum hermetic DSC pan. The sample, along with an empty reference pan, is placed in the instrument. The samples are analyzed using the following conditions/temperature program: Nitrogen Purge at a rate of 50.0 mL/min; Equilibrate @ 0.00° C.; Modulate +/−1.00° C./min every 60 seconds; until an isothermal is reach for 5.00 min; Ramp the temperature at a rate of 5° C./min to 210° C. The resulting DSC data is analyzed using TA Instruments Universal Analysis Software.

The use of differential scanning calorimetry to determine the melt transition behavior and temperature for gel networks is further described by T. de Vringer et al., Colloid and Polymer Science, vol. 265, 448-457 (1987); and H. M. Ribeiro et al., Intl. J. of Cosmetic Science, vol. 26, 47-59 (2004). The onset melt temperature is determined from the graph containing the main melt peak.

### Compression

The method can be used to determine the Compression Force of a particle. The method can be used to determine the Compression Force of a sold or swollen particle using a Kawabata™ brand KES-FB3-Auto Compression Tester from Kato Tech, LTD, or equivalent.

### Device Settings

Sensitivity: 2
Velocity: 0.02 mm/sec
Stroke: 10
Zone or Probe Compression Area: 2 (corresponds to a standard 16 mm compression probe)
Process Rate: 0.1
Maximum Load: 50 gf/cm².

Sample preparation for determining Compression Force of a swollen particle.

To prepare a swollen particles from dry, discrete particles, dry, discrete particles are placed into a receiving composition such as a shampoo or conditioner at a ratio of about 10 particles per 5 grams of composition, such that the particles are coated by the receiving composition. The particles remain in the composition for 24 hours. After 24 hours, the particles are sampled from the receiving composition one at a time using a small spatula, taking care not to deform the beads during extraction. The swollen bead is carefully centered on the sample stage of the compression tester, beneath the compression probe. Following the manufacturer's instructions for proper use of the compression tester, the Compression Force is measured.

A swollen particle that has a compression force of less than 0.5 g-f may be too soft to measure using the Compression method. In such cases, the Compression Force of the particles is recorded as <0.5 g-f.

### EXAMPLES

### Example 1: Formulations

This example provides inventive and comparative examples of discrete particles. The formulas for the particles are shown in Table 2, along with certain physical properties of the particles (i.e., acid AlogP, Onset Melt Temperature, and Creep-recovery value). Comparative examples are considered non-inventive and are designated with a “C” (e.g., C1). Inventive examples are identified as “Inv.” (e.g., Inv 1). The particles in Table 2 were made by melting the surfactant, fatty alcohol and acid together at more than 85° C., thereafter, solidifying the molten mixture in a mold of dimensions 6 mm diameter and 2 mm depth.

TABLE 2

| | Inv 1 | Inv 2 | Inv 3 |
|---|---|---|---|
| | Succinic | Maleic | Malic |
| | acid | acid | acid |
| | Wt % | | |
| SAPDMA ¹ | 28.8 | 28.84 | 28.30 |
| Succinic acid | 7.4 | — | |
| Maleic acid | — | 7.34 | |
| Malic acid | | | 8.48 |
| Cetyl alcohol | 31.9 | 31.91 | 31.61 |
| Stearyl alcohol | 31.9 | 31.91 | 31.61 |
| Acid AlogP | −0.36 | 0.04 | −1.01 |
| Onset Melt | 49.32 | 48.59 | 48.14 |
| Temp (° C.) | | | |
| Creep-recovery | −0.0020 | −0.0022 | −0.0009 |
| value @40° C. | | | |

¹ Genamin SPA from Clariant Specialty Chemicals

TABLE 2

| | C1 | C2 | C3 | C4 | C5 | C6 |
|---|---|---|---|---|---|---|
| | Wt % | | | | | |
| SAPDMA | 31.30 | 28.19 | 63.10 | 61.39 | 27.85 | 26.58 |
| L-glutamic acid | | 9.31 | | | | |
| Salicylic acid | | | 8.74 | | | |
| Citric acid | | | | 12.15 | | |
| Cinnamic acid | | | | | 9.37 | |
| Azelaic acid | | | | | | 11.90 |
| Cetyl alcohol | 34.35 | 31.25 | 14.08 | 13.23 | 31.39 | 30.76 |
| Stearyl alcohol | 34.35 | 31.25 | 14.08 | 13.23 | 31.39 | 30.76 |
| Acid AlogP | N/A | −3.46 | 1.22 | −1.32 | 1.93 | 1.92 |
| Onset Melt | 35.55 | 38.30 | 43.79 | 47.49 | 36.85 | 45.31 |
| Temp (° C.) | | | | | | |
| Creep-recovery | −0.0024 | −0.0036 | −0.0025 | −0.001 | −0.0029 | −0.0028 |
| value @40° C. | | | | | | |

### Example 2: AlogP Vs. Onset Melt Temperature Vs. Creep-Recovery

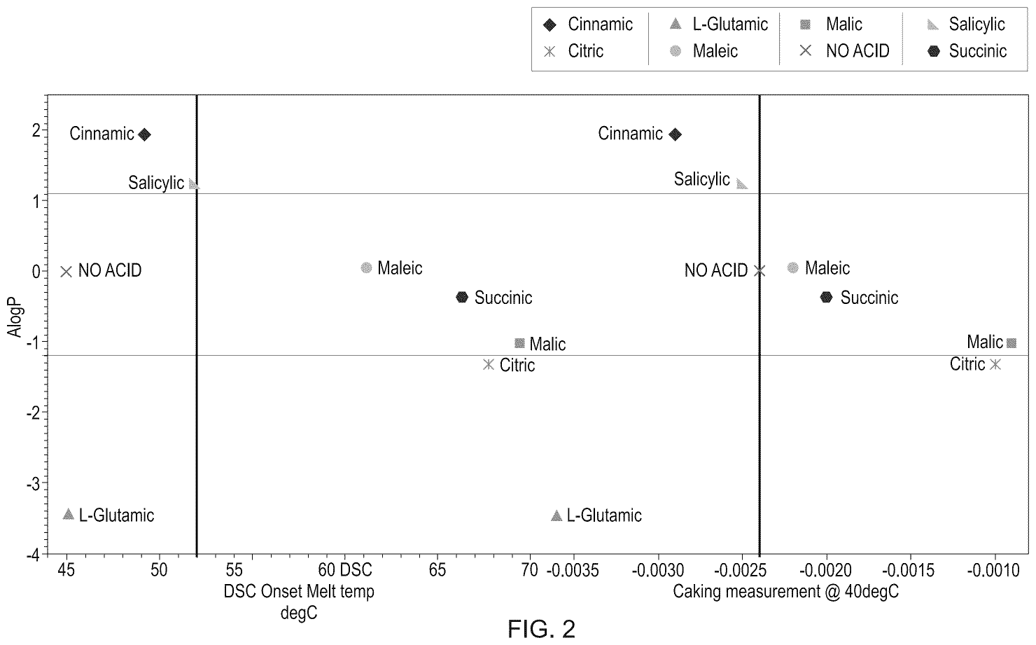

This example demonstrates the differences in physical properties when different acids are used to make the particles. Example particles C₁-C₄, Inv 3, Inv 6 and Inv 8 from Table 1 were tested according to the test methods described above to measure Creep-recovery and Onset melt temperature. The results were plotted against AlogP as shown in FIG. 2. The first vertical line 1 in FIG. 2 divides the suitable particles from the unsuitable particles, based on Onset melt temperature. The second vertical line 2 divides the suitable particles from the unsuitable particles based on the Creep-recovery. The shaded area 3 defines the characteristics of suitable particles. As can be seen in FIG. 2, only particles made with maleic acid, succinic acid or malic acid meet all the criteria for a suitable particle. These results are unexpected because it was not previously believed that these acids would behave differently from other oil soluble acids when used to formulate discrete particles.

### Examples/Combinations

1. A solid, discrete particle, comprising:
   a C₁₂-C₂₂ fatty alcohol;
   a cationic surfactant;
   an oil soluble acid having an AlogP of −1.2 to 1.1; and
   less than 50% water, preferably less than 30%, more preferably less than 10%, even more preferably less than 8% and most preferably 0%.
2. The particle of paragraph 1, further comprising a benefit agent.
3. The particle of paragraph 1 or 2, wherein the benefit agent is selected from conditioning agents, perfumes, coloring agents, sensate agents, anti-dandruff agents, incompatible agents, or a combination thereof, preferably the benefit agent is a conditioning agent selected from conditioning polymers, high melting point fatty compounds, low melting point oils, silicone compounds, and combinations thereof.
4. The particle of any preceding paragraph, wherein the particle has a longest dimension of 1 microns to 100 millimeters, preferably 10 μm to 50 mm, more preferably 50 μm to 20 mm, even more preferably 100 μm to 10 mm and even more preferably 500 μm to 5 mm.
5. The particle of any preceding paragraph, wherein the particle is anyhydrous.
6. The particle of any preceding paragraph, wherein the cationic surfactant comprises a tertiary amidoamine selected from stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide and combinations thereof, preferably the cationic surfactant is stearamidopropyl dimethylamine, behenamidopropyl dimethylamine or a combination thereof.
7. The particle of any preceding paragraph, wherein the fatty alcohol comprises stearyl alcohol, cetyl alcohol, behenyl alcohol or a combination thereof.
8. The particle of any preceding paragraph, wherein the oil soluble acid is malic acid, maleic acid, succinic acid, glyoxalic acid or a combination thereof.
9. The particle of any preceding paragraph, wherein the particle has an Onset Melt Temperature of 40° C. or greater, preferably 42° C. or greater, more preferably 44° C. or greater, and even more preferably 45° C. or greater.
10. The particle of any preceding paragraph, wherein the particle is in a shape selected from spherical, hemispherical, flake, rectangular, or fiber.
11. The particle of any preceding paragraph, wherein the particle has a Compression Force of greater than 50 g-f, according to the Compression Test.
12. The particle of any preceding paragraph, wherein the particle is free of anionic surfactant.
13. A personal care composition, comprising:
   a plurality of the discrete particles of any preceding claim; and
   an aqueous carrier, wherein the particles are dispersed in the carrier.
14. A method of making a customized personal care product, comprising:
   providing a container comprising an aqueous carrier stored therein;
   adding a plurality of the discrete particles of any one of paragraphs 1 to 12 to the aqueous carrier; and mixing the discrete particles and the aqueous carrier such that the particles are dispersed in the carrier.
15. The method of paragraph 14, wherein the particles swell in the aqueous carrier to form a semi-hydrated gel phase, and wherein the swollen particles have a compression of about 0.5 gram-force units to about 50 g-g according to the Compression Test.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid, discrete particle, comprising:
a C₁₂-C₂₂ fatty alcohol;
a cationic surfactant;
an oil soluble acid having an AlogP of −1.2 to 1.1; and
less than 50% water.

2. The particle of claim 1, further comprising a benefit agent.

3. The particle of claim 2, wherein the benefit agent is a conditioning agent, perfume, coloring agent, sensate agent, anti-dandruff agent, incompatible agent, or a combination thereof.

4. The particle of claim 3, wherein the benefit agent is a conditioning agent selected from conditioning polymers, high melting point fatty compounds, low melting point oils, silicone compounds, and combinations thereof.

5. The particle of claim 1, wherein the particle is anhydrous.

6. The particle of claim 1, wherein the particle has a longest dimension of about 1 micron to about 100 millimeters.

7. The particle of claim 1, wherein the cationic surfactant comprises a tertiary amidoamine selected from stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide and combinations thereof.

8. The particle of claim 1, wherein the cationic surfactant is stearamidopropyl dimethylamine, behenamidopropyl dimethylamine or a combination thereof.

9. The particle of claim 1, wherein the fatty alcohol comprises stearyl alcohol, cetyl alcohol, behenyl alcohol or a combination thereof.

10. The particle of claim 1, wherein the oil soluble acid is malic acid, maleic acid, succinic acid, glyoxalic acid or a combination thereof.

11. The particle of claim 1, wherein the particle has an Onset Melt Temperature of 40° C. or greater.

12. The particle of claim 1, wherein the particle is in a shape selected from spherical, hemispherical, flake, rectangular, a fiber and combinations of these.

13. The particle of claim 1, wherein the particle has a Compression Force of greater than 50 g-f, according to the Compression Test.

14. The particle of claim 1, wherein the particle is free of anionic surfactant.

15. A personal care composition, comprising:
a plurality of the discrete particles of claim 1; and
a carrier, wherein the particles are dispersed in the carrier.

16. The personal care composition of claim 15, wherein the carrier comprises more than 50% water.

17. The personal care composition of claim 15, wherein the discrete particle is in a semi-hydrated gel phase and has a compression of about 0.5 gram-force units to about 50 gram-force units according to the compression test.

18. A method of making a customized personal care product, comprising:
providing a container with an aqueous carrier stored therein;
adding a plurality of the discrete particles of claim 1 to the aqueous carrier, and
mixing the discrete particles and the aqueous carrier such that the particles are dispersed in the carrier.

19. The method of claim 18, wherein the particles swell in the aqueous carrier to form a semi-hydrated gel phase, and wherein the swollen particles have a compression of about 0.5 gram-force units to about 50 g-g according to the Compression Test.
